(19) United States
(12) Patent Application Publication
(10) Pub. No.: US 2011/0159098 A1
(43) Pub. Date: Jun. 30, 2011

(54) STABILIZATION AND DELIVERY OF NUCLEIC ACID COMPLEXES

(75) Inventor: Joram Slager, St. Louis Park, MN (US)

(73) Assignee: SurModics, Inc., Eden Prairie, MN (US)

(21) Appl. No.: 12/968,365

(22) Filed: Dec. 15, 2010

Related U.S. Application Data
(60) Provisional application No. 61/290,989, filed on Dec. 30, 2009.

(51) Int. Cl. A61K 31/713; A61K 39/395; A61K 9/00; A61K 47/48; A61P 9/00; A61P 27/02; A61P 15/00; A61P 11/00; A61P 25/00; C12N 5/071

(52) U.S. Cl. 424/484; 514/44 A; 424/130.1; 514/1.1; 435/375

(57) ABSTRACT

Compositions and methods for delivering nucleic acid, including siRNA, to a target cell are provided. In one embodiment, the composition includes the nucleic acid and a stabilizing protein. In another embodiment, the nucleic acid is complexed with a carrier, for example, a peptide carrier. In yet another embodiment, the nucleic acid combined with a protein which is cross-linked to form a proteinaceous controlled release matrix. Methods for making the compositions are also described.

### CROSS-REFERENCE TO RELATED APPLICATIONS

This application claims the benefit of U.S. Provisional Application No. 61/290,989, filed Dec. 30, 2009, the contents of which are herein incorporated by reference.

### FIELD OF THE INVENTION

The present invention relates to devices and methods for the controlled release of one or more active agents. More specifically, the present invention relates to devices and methods for the controlled release of nucleic acid active agents.

### BACKGROUND

RNA interference (RNAi) is a mechanism found in nature by which double-stranded RNA (dsRNA), when present in a cell, regulates gene expression by silencing or repressing the expression of a target gene. The double-stranded RNA responsible for inducing RNAi is termed interfering RNA. In mammalian cells, RNAi can be triggered by small interfering RNA (siRNA)

Small interfering RNA (siRNA), sometimes known as short interfering RNA or silencing RNA, is a class of double-stranded RNA molecules with a well-defined structure: a short (usually between about 18 to 25 nucleotides) double strand of RNA (dsRNA) with 2-nt 3′ overhangs on either end.

SiRNAs can be exogenously (artificially) introduced into cells by various transfection methods to knockdown a gene of interest. Essentially any gene for which the sequence is known can be targeted based on sequence complementarity with an appropriately siRNA.

Given the ability to knock down essentially any gene of interest, RNAi has generated a great deal of interest. However, in order to mediate an effect on a target cell, a nucleic acid based active agent must generally be delivered to an appropriate target cell, taken up by the cell, released from an endosome, and transported to the nucleus or cytoplasm (intracellular trafficking), among other steps. As such, successful treatment with nucleic acids depends upon site-specific delivery, stability during the delivery phase, and a substantial degree of biological activity within target cells. For various reasons, these steps can be difficult to achieve. For example, nucleic acids are readily degraded by enzymes in the in vivo environment.

### SUMMARY OF THE INVENTION

A composition for delivering small inhibitory nucleic acid (siRNA) to a target cell is described herein. In one embodiment, the composition comprises siRNA and a stabilizing protein. In a further embodiment, the stabilizing protein is selected from the group consisting of Immunoglobulin G (IgG), a fragment antigen-binding (Fab fragment), transferrin, bovine serum albumin (BSA) or human serum albumin (HSA). In one embodiment, the siRNA is complexed with a carrier. In a more particular embodiment, the carrier comprises a peptide carrier. For example, the peptide carrier can be a polycationic or amphipathic cell penetrating peptide. In particular, the peptide carrier can be N-ter and PTD-DRBD.

Also described herein is a composition for delivering small inhibitory nucleic acid (siRNA) to a target cell, wherein the composition comprises siRNA combined with a protein and the protein is cross-linked to form a proteinaceous controlled release matrix.

A method of making a composition for delivering a nucleic acid reagent to a target cell is described wherein the method comprises: combining the nucleic acid reagent, one or more stabilizing proteins, and one or more matrix forming polymers in an emulsion with a dispersed phase and a continuous phase; extracting the solvent from the sipersed phase into the extraction phase to form microparticles containing the nucleic acid reagent, one or more stabilizing proteins and one or more matrix forming proteins. In one embodiment, the nucleic acid reagent comprises siRNA. In another embodiment, the nucleic acid reagent comprises a nucleic acid complexed with a carrier. In one embodiment, the carrier comprises a peptide carrier. In another embodiment, the carrier comprises a cationic lipid carrier. In one embodiment, the stabilizing protein is selected from the group consisting of Immunoglobulin G (IgG) and a fragment antigen-binding (Fab fragment), transferrin, bovine serum albumin (BSA) and human serum albumin (HSA).

Also described are methods of delivering a small inhibitory nucleic acid (siRNA) to a target cell.

The application describes a method of making a composition for delivering a nucleic acid reagent to a target cell, the method comprising: combining nucleic acid reagent with a protein in an aqueous solution; adding a phase separation agent to the aqueous solution, wherein the phase separation agent causes the siRNA and protein to precipitate out of solution to form particles; and cross-linking the protein to form a proteinaceous controlled release matrix. In one embodiment, the nucleic acid reagent comprises siRNA. In another embodiment, the nucleic acid reagent comprises a nucleic acid complexed with a carrier. In a more particular embodiment, the carrier comprises a peptide carrier. In one embodiment, the peptide carrier comprises N-ter.

### BRIEF DESCRIPTION OF THE DRAWINGS

The invention may be more completely understood and appreciated in consideration of the following detailed description of various embodiments of the invention in connection with the accompanying drawings.

FIG. 1 is a graph showing siRNA/DOTAP complexes with added proteins and PEG-phase separation.

FIG. 2 is a graph showing siRNA/N-ter complexes with added proteins and PEG-phase separation.

FIG. 3 is a graph showing subsequent suspension in solvents of N-ter/siRNA complexes in protein particles by PEG-phase separation.

FIG. 4 is a graph showing the influence of emulsifying on transfection.

FIG. 5 is a graph showing the effect of the DOTAP/Cholesterol to siRNA ratio on transfection efficacy.

FIG. 6 is a graph showing the effect of protein on transfection efficiency.

### DETAILED DESCRIPTION

The invention described herein generally relates the use of nucleic acids as bioactive agents. In one embodiment, the nucleic acid includes small interfering RNA (siRNA), which can be used to control expression of a gene of interest. In particular, the invention provides compositions and methods for increasing the stability and/or transfection efficiency of a nucleic acid.

### Nucleic Acids

Nucleic acids used with embodiments of the invention can include various types of nucleic acids including those that can function to provide a therapeutic effect. Although the disclosure focuses on ribonucleic acids (RNA), in particular, small interfering RNA (siRNA), the concepts herein can be applied to other nucleic acids including, but not limited to, deoxyribonucleic acids (DNA), micro RNA (miRNA), piwi-interacting RNA (piRNA), short hairpin RNA (shRNA), antisense nucleic acids, locked nucleic acids and catalytic DNA. As discussed in more detail below, a nucleic acid may or may not be complexed with a carrier. The term “nucleic acid reagent” as used herein is intended to encompass a nucleic acid with or without a carrier.

In one embodiment, the nucleic acid includes small interfering RNA (siRNA), also known as short interfering RNA or silencing RNA. In one embodiment, the siRNA is capable of reducing or inhibiting expression of a target gene (e.g., by mediating the degradation of mRNAs which are complementary to the sequence of the interfering RNA) when the siRNA is in the same cell as the target gene. As used herein the term “target gene” refers to a gene whose expression is to be selectively inhibited or “silenced” by the siRNA. Silencing of the target gene is achieved by cleaving the mRNA of the target gene by an siRNA, e.g., an isolated siRNA or one that is created from an engineered RNA precursor.

In one embodiment, the siRNA includes a duplex, or double-stranded region, of about 18-25 nucleotides long. Often siRNAs contain from about two to about four unpaired nucleotides at the 3′ end of each strand. Typically, at least one strand of the duplex or double-stranded region of a siRNA is substantially homologous to or substantially complementary to a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. The siRNA can be formed as a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule.

In one embodiment, siRNA are chemically synthesized. In an alternate embodiment, siRNA are generated by cleavage of longer dsRNA (e.g., dsRNA greater than about 25 nucleotides in length), for example, with an enzyme such as E. coli RNase III or Dicer.

### Carrier Agent

Unmodified, naked nucleic acid, such as siRNAs, tend to be relatively unstable in blood and serum, as may be rapidly degraded by endo- and exonucleases, resulting in short half-lives in vivo. Additionally, the relatively large molecular weight (˜13 kDa) and polyanionic nature (˜40 negative phosphate charges) of naked nucleic acids such as siRNA make it difficult for the molecule to freely cross the cell membrane. Carrier agents can be used to increase cellular accumulation of exogenously delivered nucleic acid molecules and to facilitate release from endosomes to the cytosol.

In many instances, access to the intracellular matrix is gained by complexing the nucleic acid of interest to a carrier agent. As used herein, the term “complex” or “complexing” refers to a chemical association of two or more chemical species through non-covalent bonds such as electrostatic interactions, hydrogen bonding interactions, and hydrophobic interactions. The term “nucleic acid complex” refers in general to a nucleic acid active agent and one or more carrier agents complexed to the nucleic acid. In one embodiment, the carrier can condense and protect the nucleic acid, target specific cells or tissues, and/or can help mediate intracellular delivery of the nucleic acid. In one embodiment, the nucleic acid complex includes siRNA complexed to one or more carrier agents. As used herein, the term “carrier agent” refers to compounds than can be complexed with nucleic acids to preserve the activity of the nucleic acids during manufacturing and/or delivery. Many types of carrier agent are known, and include, for example, charged compounds, for example, cationic compounds (i.e., compounds having a net positive charge) and charge neutral compounds. More specifically, carrier agents can include cationic polymers that are capable of efficiently condensing nucleic acids into nanoparticles, termed “polyplexes,” by self-assembly via electrostatic interactions. Sometimes the cationic polymer includes one or more functional groups that can be modified with ligands, such as cell-targeting molecules. Examples of cationic polymers include, but are not limited to poly-L-lysine (PLL) and poly(ethylenimine) (PEI). Another class of carrier includes cationic lipids. Cationic lipid carriers are commercially available and include, but are not limited to 1,2-dioleoyl-3-trimethylammonium propane (DOTAP), N-methyl-4-(dioleyl)methylpyridinium (SAINT-2), 3β-[N—(N′,N′-dimethylaminoethane)-carbamoyl]cholesterol (DC-Chol), or a gemini surfactant (e.g., a surfactant having two conventional surfactant molecules chemically bonded together by a spacer), such as GS1 (a sugar-based gemini surfactant), as well as the neutral lipid dioleoylphosphatidylethanolamine (DOPE) and cholesterol. Addition of polyanionic nucleic acids to mixtures of cationic lipids or liposomes results in the self-assembly of particles termed “lipoplexes.” Other classes of carrier molecule include polypeptides and nanoparticles, such as gold nanoparticles. In one embodiment, the carrier can be conjugated to one or more molecules that target specific cell types. Examples of targeting agents include antibodies and peptides which recognize and bind to specific cell surface molecules.

Typically, nucleic acid/carrier complexes self-assemble when brought into contact with one another, for example, in an aqueous solution. For example, a complex may form due to the charge-charge interactions between a negatively charged nucleic acid and a positively charged carrier agent. In some instances, particles—either micelles or liposomes with lipid bilayers—can be formed when the nucleic acid interacts with the positively charged transfection agents.

### Peptide Carriers

The cellular membrane provides a barrier that protects the intracellular environment of the cell. Passage of molecules across this barrier is highly regulated and highly restricted. However, nucleic acids such as siRNA can be conjugated to one or more peptide carriers to enhance the efficiency of transport of the siRNA into living cells compared to the efficiency of delivery to unmodified siRNA. In one embodiment, the carrier includes a cell penetrating peptide (CPPs), which can also be referred to as protein transduction domains (PTDs). Various CPPs are known and have been used for the delivery of a wide range of macromolecules including peptides, proteins and antisense oligonucleotides. In one embodiment, the carrier includes a CCP complexed with siRNA to deliver the siRNA into cells.

The term “cell-penetrating peptide (CPP)” refers to short peptides, typically less than 30 amino acids in length, which are able to penetrate cell membranes and translocate covalently coupled cargo into the cell. In general, CCPs are amphipathic and have a net positive charge. The term “amphipathic” means that the molecule includes both hydrophobic and hydrophilic regions.

Other peptide carriers include engineered polypeptides having one or more functionalities, including, but not limited to: nucleic acid condensation, targeting, endosomal escape, and nuclear entry. Examples of peptide carriers include, but are not limited to N-ter (a cationic peptide siRNA delivery vehicle available from Sigma (St. Louis, Mo.) and PTD-DRBD, a fusion protein comprising cell penetrating domains and double-stranded RNA binding domains. (developed by Traversa Therapeutics, Inc. San Diego, Calif.).

### Stabilizing Proteins

In one embodiment, the nucleic acid is combined with one or more stabilizing proteins to form a nucleic acid/stabilizing protein complex. In another embodiment a nucleic acid/carrier complex is combined with a stabilizing protein to form a nucleic acid/carrier/stabilizing protein complex. As used herein, the term “stability” or “stabilized” refers to the ability of the nucleic acid to resist degradation (enzymatic, mechanical and/or chemical), dissociation and/or inactivation while maintaining biological activity, for example, in the case of siRNA, biological activity can refer to the ability to knockdown a gene of interest. Stability can be measured in terms of half-life (e.g., the time taken for the 50% of the nucleic acid to be degraded). As used herein, the term “stabilizing protein” refers to a protein that improves the ability of a nucleic acid such as siRNA to resist degradation and retain transfection efficiency. In particular, it has been found that the inclusion of one or more stabilizing proteins can improve stability of the nucleic acid/carrier complex in an emulsion, for example, in an organic solvent during the generation of polymeric controlled release microparticle (see below).

In one embodiment, the stabilizing protein includes an immunoglobulin or antibody. As used herein, the terms “immunoglobulin” or “antibody” refer to proteins that bind a specific antigen. Immunoglobulins generally have two identical heavy chains and two light chains, although the terms antibody and immunoglobulin can also encompass single chain antibodies and two chain antibodies. Examples of immunoglobulins include, but are not limited to, polyclonal, monoclonal, chimeric, and humanized antibodies, Fab fragments, F(ab′)₂ fragments, and immunoglobulins of the following classes: IgG, IgA, IgM, IgD, IgE, and secreted immunoglobulins (sIg). In a more particular embodiment, the stabilizing protein includes Immunoglobulin G (IgG) or a fragment antigen-binding (Fab) fragment.

In another embodiment the protein can include one or more blood plasma proteins, such as albumin, for example, human serum albumin (HSA) or bovine serum albumin (BSA), or a protein that targets cell-membrane receptors, for example glycoproteins such as transferrin (also called apo-transferrin, when not bound to iron). In one embodiment, transferrin is used as a targeting agent in connection with Calando's transfection agent (Calando Pharmaceuticals, Inc., Pasadena, Calif.), a cyclodextrin-containing polycation.

The inventors have found that the inclusion of one or more proteins along with a nucleic acid or a nucleic acid/carrier complex during the formation of polymeric controlled release microparticles can increase the stability of the nucleic acid, particularly in an emulsion. In particular, inclusion of one or more stabilizing proteins along with an siRNA/carrier complex, such as an siRNA/carrier protein complex, results in increased transfection efficiency. In contrast, emulsifying an aqueous solution of nucleic acid/carrier complex in an organic solvent in the absence of a stabilizing protein resulted in a reduction or loss of transfection activity.

### Polymeric Microparticles

Polymeric microparticles can be used to provide controlled release of many different types of active agents. In one embodiment, polymeric microparticles can be used for the controlled release of naked nucleic acids (i.e., without a carrier or stabilizing protein). In another embodiment, the nucleic acid can be a part of a nucleic acid/carrier complex. In a more particular embodiment, the nucleic acid is siRNA and the carrier is a protein carrier. In yet another embodiment, the nucleic acid can be included in a nucleic acid/stabilizing protein complex. In another embodiment the nucleic acid can be included in a nucleic acid/carrier/stabilizing protein complex.

Applicants have found that nucleic acid complexes disposed within polymeric microparticles are more robust and less subject to degradation during processing, retain their activity and can be used successfully to transfect target cells (See, U.S. patent application Ser. No. 12/437,287, filed May 7, 2009, entitled DELIVERY OF NUCLEIC ACID COMPLEXES FROM PARTICLES, the disclosure of which is hereby incorporated by reference in its entirety). However, one challenge associated with delivery of nucleic acid complexes from polymeric microparticles, is the dissociation and/or inactivation of the nucleic acids under conditions typically used for matrix formation. For example, nucleic acid complexes may become inactivated or otherwise damaged by organic solvents commonly used to form polymeric microparticles.

In one embodiment, the nucleic acid reagent (i.e., with or without a carrier) is encapsulated within a polymeric microparticle. Methods for forming polymeric microparticles are known and include, for example, emulsion-based process, such as, oil/water, water/oil, water/oil/water, or oil/water/oil processes. In general, emulsion-based processes involve the preparation one or more dispersions. Although there are three main types of dispersions (suspensions, colloids and solutions), a dispersion can generally be described in terms of a first phase and a second phase wherein the first phase, known as the “dispersed phase” or “dispersed phase solution,” is discontinuous in the second phase, known as the “continuous phase” or “continuous phase solution.” Once formed, the emulsion is diluted with additional solvent or solution, known as the “extraction phase” or “extraction solution.” In general, the solvent used for the dispersed phase is soluble in the extraction phase, although some solvents are more soluble in the extraction phase or extraction solution than others. In one embodiment, the dispersion includes a siRNA-complex in solid particles that is suspended in a polymer solution, which in turn is emulsified in a continuous phase. In another embodiment, the dispersion includes a siRNA-complex in water, which is first emulsified in polymer solution, which in then emulsified in a continuous phase.

To form a polymeric microparticle, a matrix-forming polymer is dissolved or dispersed in a solvent to form a dispersed phase or a dispersed solution. The dispersed phase is then combined with the continuous phase to form an emulsion. Subsequently, the dispersed phase/continuous phase emulsion is combined with the extraction phase, wherein solvent from the dispersed phase is extracted into the extraction phase, resulting in the hardening of the discontinuous droplets of the dispersed phase to form polymer-rich microparticles. A wide variety of polymers can be used as matrix-forming polymers, including homopolymers, copolymers, and polymer mixtures, discussed in more detail below.

In one embodiment, the nucleic acid reagent is dissolved or suspended in an aqueous solvent prior to incorporation into a polymeric microparticle. In general, naked siRNA is soluble in water, whereas when complexed with a carrier, the siRNA complex forms nano-sized particles However, in other embodiments, the nucleic acid reagent can be suspended in an organic solvent. However, while not intending to be bound by theory, it is believed that the use of some organic solvents to suspend the nucleic acid delivery particles can contribute to degradation of the particles, damaging DNA, and/or aggregation of the particles.

### Matrix-Forming Polymers

As described above, the nucleic acid reagent can be included in a controlled release polymeric microparticle. Various matrix-forming polymers can be used to form the controlled release polymeric microparticle. Examples of polymers that can be used to form a particle that can include nucleic acids include ethylene vinyl alcohol copolymer; poly(hydroxyvalerate); poly(L-lactic acid); polycaprolactone; poly(lactide-co-glycolide); poly(hydroxybutyrate); poly(hydroxybutyrate-co-valerate); polydioxanone; polyorthoester; polyanhydride; poly(glycolic acid); poly(D,L-lactic acid); poly(glycolic acid-co-trimethylene carbonate); polyphosphoester; polyphosphoester urethane; poly(amino acids); cyanoacrylates; poly(trimethylene carbonate); poly(iminocarbonate); copoly(ether esters) (e.g., PEO/PLA); polyalkylene oxalates; polyphosphazenes; biomolecules, such as fibrin, fibrinogen, cellulose, starch, collagen and hyaluronic acid; polyurethanes; silicones; polyesters; polyolefins; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrilestyrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; and carboxymethyl cellulose.

### Cross-Linking

In one embodiment, one or more proteins are combined with a nucleic acid or a nucleic acid/carrier complex in an aqueous solution, wherein the nucleic acid serves as a nucleating agent and a proteinaceous particle is formed around the nucleic acid or nucleic acid/carrier complex, for example by phase separation. In a phase separation process, a particle can be formed by the addition of an amphiphilic polymer to the aqueous solution, which results in the precipitation of the nucleic acid or nucleic acid/transfection agent complex and proteins from solution to form proteinaceous microparticles. Phase separation is described more fully in co-pending U.S. patent application Ser. No. 12/437,287, filed May 7, 2009, entitled DELIVERY OF NUCLEIC ACID COMPLEXES FROM PARTICLES, the disclosure of which is hereby incorporated by reference in its entirety.

In one embodiment, a nucleic acid reagent (i.e., with or without a carrier) can be combined with one or more proteins in a phase separation process, wherein the proteins can then be cross-linked to form a proteinaceous controlled release matrix. If desired the cross-linked protein complex (i.e., the nucleic acid and proteinaceous controlled release matrix) can be included within a polymeric controlled release matrix or polymeric microparticle.

A wide variety of proteins are suitable for use as a proteinaceous controlled release matrix. As used herein, the term “protein,” or “polypeptide,” refers to polymers of amino acid monomers joined together by the peptide bonds (also called peptide or amide linkages) between the carboxyl and amino groups of adjacent amino acid residues. Particularly useful proteins include proteins that are capable of stabilizing the protein, without interfering with the activity of the siRNA-transfection agent complex. Examples of suitable proteins include, for example, immunoglobulins, such as IgG or Fab, albumins such as bovine serum albumin (BSA) or human serum albumin (HSA), collagen or collagen derivatives such as gelatin and/or proteins with cell targeting potential, including proteins (or ligands) that recognize and/or bind to cell surface receptors, including but not limited to transferrin.

As used herein, the term “cross-linking” refers to one or more physical linkages between polymer chains. In one embodiment, the polymer is a protein. The physical cross-linking can be the result of one or more covalent bonds formed within a single polymer chain or across two or more polymer chains. In one embodiment, crosslinking occurs by the formation of one or more covalent bond between one or more uncharged amino groups present on the protein.

There are many known reagents that are capable of crosslinking amine functional groups of biopolymers such as proteins or polypeptides. Homobifunctional amine crosslinkers include aldehyde-based crosslinker such as glutaraldehyde, or other crosslinkers such as genipin. Alternately, cross-linking between two amine-containing molecules can be accomplished by thiolating one or more amines on one of the biomolecules and converting one or more amines on the second biomolecule to a thiol-reactive functional group such as a maleimide or iodoacetamide. It is believed that the degree of cross-linking can affect particle size and release properties of the nucleic acid encapsulated in the proteinaceous matrix. In general, the degree of crosslinking can be controlled by changing the ratio of reactants, varying the number of reactive groups on the cross-linker (e.g., using a crosslinker with more than one reactive group) or by varying the reaction time (determined by the time at which quencher is added to the reaction).

In a phase separation process, the nucleic acid or nucleic acid/carrier is combined with a protein in a solution at a concentration of at least about 10 mg/ml, generally between about 10 mg/ml and 50 mg/ml. Phase separation, and concomitant particle formation, is caused by the addition of a phase separation agent to the solution to form particles. The phase separation agent can be a polymeric or non-polymeric amphiphilic compound. Examples of suitable amphiphilic polymers include, but are not limited to, poly(ethylene glycol) (PEG) and PEG copolymers, tetraethylene glycol, triethylene glycol, trimethylolpropane ethoxylate, pentaeerythritol ethoxylate, polyvinylpyrrolidone (PVP) and PVP copolymers, dextran, Pluronic™, polyacrylic acid, polyacrylamide, polyvinyl pyridine, polylysine, polyarginine, PEG sulfonates, fatty quaternary amines, fatty sulfonates, fatty acids, dextran, dextrin, and cyclodextrin. The amphiphilic polymer can also include copolymers of hydrophilic and hydrophobic polymer blocks. The phase separation agent can be added to the solution such that the final concentration of the amphiphilic reagent is at least about 1% (w/v), or between about 2.5% (w/v) and about 12.5% (w/v), or between about 5% (w/v) and about 10% (w/v).

### Controlled-Release Coatings

The nucleic acid reagent, microparticle and/or cross-linked protein complex can also be incorporated into a controlled release coating or matrix as described in U.S. patent application Ser. No. 12/353,792, filed Jan. 14, 2009, entitled DEVICES AND METHODS FOR ELUTION OF NUCLEIC ACID DELIVERY COMPLEXES, the disclosure of which is incorporated by reference in its entirety.

### Methods and Devices

The invention described herein can be used for transfection of a target cell with a nucleic acid of interest, including, for example, therapeutic administration of nucleic acids to a patient in need thereof. In one embodiment, the invention described herein is useful to knockdown, reduce or inhibit expression of a target gene. As used herein, the term “inhibit” refers to a decrease in the level of gene expression or RNA function as the result of interference with or interaction with gene expression or RNA function as compared to the level of expression or function in the absence of the interference or interaction. The inhibition may be complete, in which there is no detectable expression or function, or it may be partial. Partial inhibition can range from near complete inhibition to near absence of inhibition; typically, inhibition is at least about 50% inhibition, or at least about 80% inhibition, or at least about 90% inhibition. The term “transfection” refers to the introduction of foreign nucleic acids into cells and may be accomplished by a variety of means known to the art.

The invention described herein is suitable for both systemic and local administration of nucleic acid bioactive agents. The embodiments described herein can include and can be used with (such as disposed on the surface of) implantable, or transitorily implantable, devices including, but not limited to, vascular devices such as grafts (e.g., abdominal aortic aneurysm grafts, etc.), stents (e.g., self-expanding stents typically made from nitinol, balloon-expanded stents typically prepared from stainless steel, degradable coronary stents, etc.), catheters (including arterial, intravenous, blood pressure, stent graft, etc.), valves (e.g., polymeric or carbon mechanical valves, tissue valves, valve designs including percutaneous, sewing cuff, and the like), embolic protection filters (including distal protection devices), vena cava filters, aneurysm exclusion devices, artificial hearts, cardiac jackets, and heart assist devices (including left ventricle assist devices), implantable defibrillators, electro-stimulation devices and leads (including pacemakers, lead adapters and lead connectors), implanted medical device power supplies (e.g., batteries, etc.), peripheral cardiovascular devices, atrial septal defect closures, left atrial appendage filters, valve annuloplasty devices (e.g., annuloplasty rings), mitral valve repair devices, vascular intervention devices, ventricular assist pumps, and vascular access devices (including parenteral feeding catheters, vascular access ports, central venous access catheters); surgical devices such as sutures of all types, staples, anastomosis devices (including anastomotic closures), suture anchors, hemostatic barriers, screws, plates, clips, vascular implants, tissue scaffolds, cerebro-spinal fluid shunts, shunts for hydrocephalus, drainage tubes, catheters including thoracic cavity suction drainage catheters, abscess drainage catheters, biliary drainage products, and implantable pumps; orthopedic devices such as joint implants, acetabular cups, patellar buttons, bone repair/augmentation devices, spinal devices (e.g., vertebral disks and the like), bone pins, cartilage repair devices, and artificial tendons; dental devices such as dental implants and dental fracture repair devices; drug delivery devices such as drug delivery pumps, implanted drug infusion tubes, drug infusion catheters, and intravitreal drug delivery devices; ophthalmic devices including orbital implants, glaucoma drain shunts and intraocular lenses; urological devices such as penile devices (e.g., impotence implants), sphincter, urethral, prostate, and bladder devices (e.g., incontinence devices, benign prostate hyperplasia management devices, prostate cancer implants, etc.), urinary catheters including indwelling (“Foley”) and non-indwelling urinary catheters, and renal devices; synthetic prostheses such as breast prostheses and artificial organs (e.g., pancreas, liver, lungs, heart, etc.); respiratory devices including lung catheters; neurological devices such as neurostimulators, neurological catheters, neurovascular balloon catheters, neuro-aneurysm treatment coils, and neuropatches; ear nose and throat devices such as nasal buttons, nasal and airway splints, nasal tampons, ear wicks, ear drainage tubes, tympanostomy vent tubes, otological strips, laryngectomy tubes, esophageal tubes, esophageal stents, laryngeal stents, salivary bypass tubes, and tracheostomy tubes; biosensor devices including glucose sensors, cardiac sensors, intra-arterial blood gas sensors; oncological implants; and pain management implants.

In some aspects, embodiments of the invention can include and be utilized in conjunction with ophthalmic devices. Suitable ophthalmic devices in accordance with these aspects can provide bioactive agent to any desired area of the eye. In some aspects, the devices can be utilized to deliver bioactive agent to an anterior segment of the eye (in front of the lens), and/or a posterior segment of the eye (behind the lens). Suitable ophthalmic devices can also be utilized to provide bioactive agent to tissues in proximity to the eye, when desired.

In some aspects, embodiments of the invention can be utilized in conjunction with ophthalmic devices configured for placement at an external or internal site of the eye. Suitable external devices can be configured for topical administration of bioactive agent. Such external devices can reside on an external surface of the eye, such as the cornea (for example, contact lenses) or bulbar conjunctiva. In some embodiments, suitable external devices can reside in proximity to an external surface of the eye.

Devices configured for placement at an internal site of the eye can reside within any desired area of the eye. In some aspects, the ophthalmic devices can be configured for placement at an intraocular site, such as the vitreous. Illustrative intraocular devices include, but are not limited to, those described in U.S. Pat. Nos. 6,719,750 B2 (“Devices for Intraocular Drug Delivery,” Varner et al.) and 5,466,233 (“Tack for Intraocular Drug Delivery and Method for Inserting and Removing Same,” Weiner et al.); U.S. Publication Nos. 2005/0019371 A1 (“Controlled Release Bioactive Agent Delivery Device,” Anderson et al.), 2004/0133155 A1 (“Devices for Intraocular Drug Delivery,” Varner et al.), 2005/0059956 A1 (“Devices for Intraocular Drug Delivery,” Varner et al.), and 2003/0014036 A1 (“Reservoir Device for Intraocular Drug Delivery,” Varner et al.); and U.S. application Ser. Nos. 11/204,195 (filed Aug. 15, 2005, Anderson et al.), 11/204,271 (filed Aug. 15, 2005, Anderson et al.), 11/203,981 (filed Aug. 15, 2005, Anderson et al.), 11/203,879 (filed Aug. 15, 2005, Anderson et al.), 11/203,931 (filed Aug. 15, 2005, Anderson et al.); and related applications.

In some aspects, the ophthalmic devices can be configured for placement at a subretinal area within the eye. Illustrative ophthalmic devices for subretinal application include, but are not limited to, those described in U.S. Patent Publication No. 2005/0143363 (“Method for Subretinal Administration of Therapeutics Including Steroids; Method for Localizing Pharmacodynamic Action at the Choroid and the Retina; and Related Methods for Treatment and/or Prevention of Retinal Diseases,” de Juan et al.); U.S. application Ser. No. 11/175,850 (“Methods and Devices for the Treatment of Ocular Conditions,” de Juan et al.); and related applications.

### EXAMPLES

### Example 1

### Addition of Proteins and Maltodextrane Derivatives to Nter/siRNA Complexes in the Formation of Discrete Particles by Phase-Separation

### Background

Previous studies, for example, those found in U.S. patent application Ser. No. 12/437,287, filed May 7, 2009, entitled DELIVERY OF NUCLEIC ACID COMPLEXES FROM PARTICLES have demonstrated that siRNA/N-ter complexes can be encapsulated in IgG, Fab or BSA particles using phase-separation methodology (see, e.g., Examples 9-12). In the studies described therein, various excipients, including protein solutions containing IgG/Fab were added to a solution containing siRNA/N-ter complexes. Phase-separation was achieved by adding a 30% w/w polyethylene glycol (PEG) 20 kDa solution. The results demonstrated that the particles possess a somewhat increased transfection efficacy compared to control solutions. Additionally, it was shown that isolated particles could be suspended in organic solvents without losing transfection efficiency.

In some cases a binary aqueous system is obtained (two immiscible water phases), but under certain conditions the macromolecules will phase-separate in such a way that solid particles are formed. In previous research (See, Examples 1-3 of U.S. patent application Ser. No. 12/437,287) it was shown that additional DNA/PEI complex particles would end up in these phase-separated particles and little or no PEI/DNA could be found in the PEG phase.

### Aim

In these experiments, protein excipients such as BSA, HSA, Fab, and IgG, were investigated for their ability to encapsulate and stabilize siRNA complexes during emulsification in organic solvents.

### Methods

siRNA Transfection Complex Preparation

50 μl samples of siRNA complexes were prepared using either anti-luciferase siRNA or scrambled siRNA and N-ter or DOTAP/Cholesterol as transfection agents:
(a) siRNA/N-ter: 1.5 μl siRNA 20 uM was diluted in 45 μl N-ter buffer. 3.75 μl Nter was added and vortexed.
(b) siRNA/DOTAP/Cholesterol: 1.5 μl siRNA 20 uM was diluted in 45 μl distilled deionized water (DDW). 4.2 μl DOTAP/Cholesterol (9:1 ratio, 1 mg/ml solution in ethanol) was added and vortexed.

Typically larger amounts of siRNA and transfection agents in buffer were mixed and the resulting siRNA/Nter or siRNA/DOTAP-Cholesterol complexes were then divided in 50 μl portions.

### Excipient Addition and Phase Separation

For some experiments Fab in phosphate buffered saline (PBS) (Southern Biotech) or IgG (lyophilized, Lampire, reconstituted in slightly acidic PBS with drop of HCl), were put on a BioRad™ desalting column and eluted with 10 mM phosphase (Ps)/no NaCl. Using a centrifuge filter the protein solutions were concentrated to approximately 40 mg/ml.

To the siRNA complex samples 50 μl of the following solutions was added:
bovine serum albumin (BSA) at 40 mg/ml
IgG at 40 mg/ml in 10 mM Ps (phosphase, no NaCl)
Fab at 40 mg/ml in 10 mM Ps (phosphase, no NaCl)

A solution containing 400 μl polyethylene glycol (PEG) 30% w/w in distilled deionized water (DDW) was added and the resulting mixture was thoroughly vortexed until cloudiness occurred. The resulting particles were treated in one of the following ways:
1. The phase-separated particles were spun down (2500 rpm, 15 minutes) and the polyethylene glycol (PEG) solution was removed.
   a. The resulting particles were reconstituted in cell medium; or
   b. The resulting particles were briefly washed with isopropyl alcohol (IPA) to remove residual water and then suspended in dichloromethane (DCM). The suspension was homogenized (IKA25T, 10 krpm for 1 minute) and solvent was evaporated in a vacuum; or
2. The siRNA complex/PEG mixture was frozen on dry ice and lyophilized. The PEG-cake was re-dissolved in dichloromethane. Particles were isolated by centrifugation.

0.4 ml of various solvents were added to the dried particles, vortexed thoroughly and/or sonicated in a sonicator. The solvents used were:
Dichloromethane
Chloroform
EthylAcetate
Cyclohexane

Subsequently, the organic phase was removed and evaporated under vacuum. 600 μl DMEM (with FBS and 5 μg/ml doxycyclin) was added to the resulting residues and vortexed thoroughly (resulting in siRNA at 50 nM concentration).

### Cell Testing

For samples containing N-ter complexes, Dulbecco's Modified Eagle Medium (DMEM) (GIBCO)/10% w/v fetal bovine serum (FBS) was added to the media with transfection complexes and left for 24 hours. For samples containing DOTAP/Cholesterol as transfection agent, serum-free DMEM was used. 5 μg/ml doxycyclin (dox) was added to all media in order to induce the HR5CL11 cells (doxyclin-induced luciferase expressing cells) to express luciferase. Cell medium was added to the various siRNA complex containing samples maintaining a siRNA concentration of 50 nM. 1000 of the 50 nM siRNA solution was added to 4 wells of a 96-well plate that was seeded with HR5CL11 cells at 10⁵ cell/ml and incubated for 24 hours prior to the transfection. The remaining solution was diluted with media to obtain siRNA at 25 nM concentration of which again 100 μl was added to 4 wells.

When initially incubated with serum-free medium, the cell media was replaced with fresh DMEM/10% FBS/5 μg/ml dox after 3 hours and further incubated for 24 hours.

The cells were then treated with Cell Titre Blue™ (Promega) to assess any toxicity. Upon lysing with Bright-Glo™ lysis buffer (Promega) luciferase content was measured by luminescence.

### Data Processing

It is known that HR5CLII cells express luciferase in a stable manner in the presence of doxycycline. Thus, effective gene-knock down should result in a reduction in luciferase expression.

The raw data obtained from luciferase assay was normalized for any toxicity using the following formula:

ND=CL/Abs*5000

ND=normalized data

CL=chemiluminescence (relative light units/RLU)

Abs=absorption of Cell Titre Blue

Gene knock-down was expressed as 100%−RLU_{luc}/RLU_{control}*100%. RLU=relative light units.

### Results

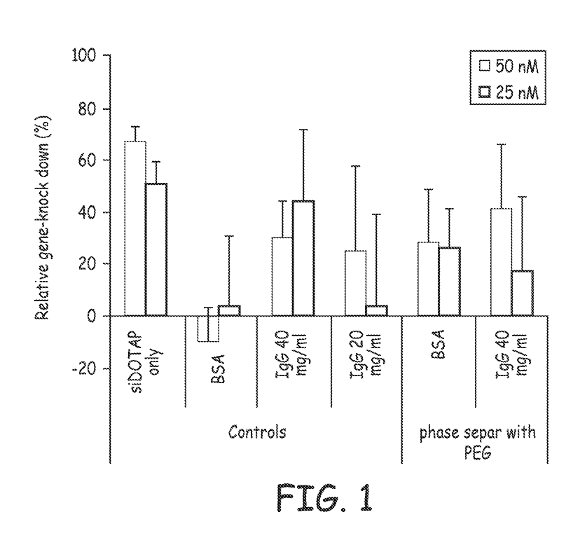

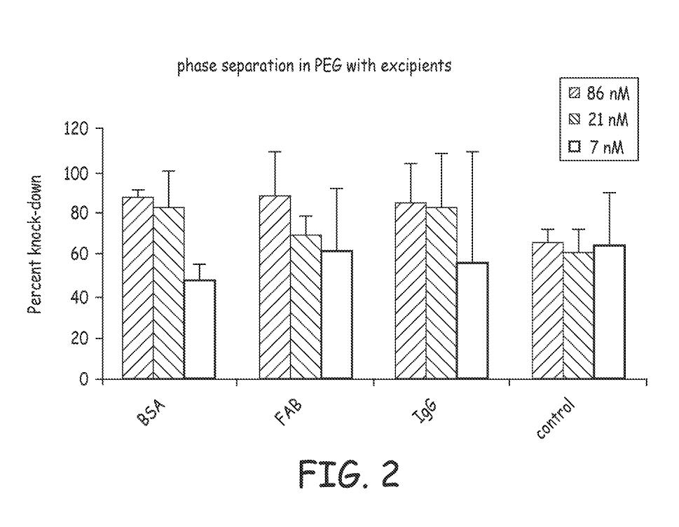

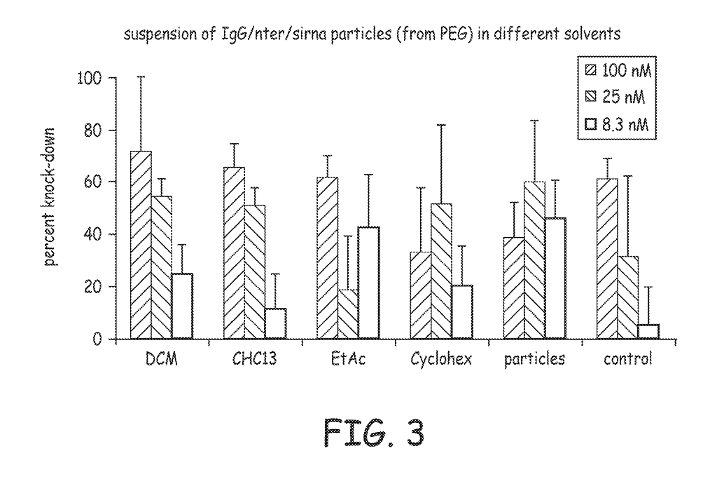

Bioactivity was plotted as the percentage of gene-knockdown. FIGS. 1 and 2 show the effect of incorporating siRNA/DOTAP/Cholesterol or siRNA/N-ter complexes respectively, in protein particles using PEG-phase separation versus control samples (where protein is added to the aqueous samples without subsequent phase separation). After isolating siRNA/N-ter complex containing phase separated particles, bioactivity of the complexes was retained even after they were resuspended in various solvents as shown in FIG. 3.

The results indicate that not only is close to all of the siRNA/Nter complex incorporated in the protein rich phase, but also that the efficacy has remained equal or has surpassed that of the control. When IgG was used for particle formation, efficacy was retained upon suspension in common solvents such as DCM and EthylAcetate. Although at 25 nM mixed results were obtained, the increased values for gene-knockdown at 8.3 nM were observed.

### Example 2a

### Cross-Linking

Upon phase separation of a solution of siRNA/N-ter complexes and IgG, the isolated particles are lyophilized and resuspended in anhydrous DCM. The protein matrix of the particles can subsequently be crosslinked by adding a solution of alkyldicarbonylimidazole, PEG-dicarbonylimidazole and stirring for one hour. Alternatively crosslinking can be accomplished by adding a solution of genipin in methanol with stirring over night. In a third method, an alkyldiisocyanate is added and stirring is only necessary for about 1 hour. The mixture is pipetted onto a PTFE centrifuge filter (0.2 um) and the particles are washed with additional DCM. Crosslinking is tested by resuspension in PBS.

The crosslinked complex can be used as an injectable controlled release matrix, or they can be incorporated into polymeric microparticles as described in U.S. patent application Ser. No. 12/437,287, filed May 7, 2009, entitled DELIVERY OF NUCLEIC ACID COMPLEXES FROM PARTICLES or included in a controlled release matrix U.S. patent application Ser. No. 12/353,792, filed Jan. 14, 2009, entitled DEVICES AND METHODS FOR ELUTION OF NUCLEIC ACID DELIVERY COMPLEXES. The disclosures of both applications are hereby incorporated by reference herein.

### Example 2b

### Inclusion of siRNA/Complex Containing Protein Particles in a Polymeric Matrix

siRNA/N-ter complexes are formed as described in the previous Examples, for example, by combining 1.5 μl of 20 μM siRNA with 3.75 μl N-ter in 50 μl N-ter specific buffer and vortexing. A solution containing a stabilizing protein, for example, 50 μl of a 40 mg/ml solution of Fab is then added to a 50 μl sample of siRNA to form a siRNA/N-ter/stabilizing protein complex. PEG 20 kDa at 30% w/w is added until the solution turns turbid indicating the phase separation. To this aqueous biphasic system 0.25% gluteraldehyde in 30% PEG 20 kDa is added and stirred for 10 to 30 minutes. Unreacted glutaraldehyde is quenched by adding ethanolamine (1 M). After stirring the mixture for 30 minutes, the particles are spun and the supernatant is discarded.

In one example, 50 mg of phase separated protein particles are mixed with 5 ml PLGA 50/50 solution in DCM and homogenized IKA probe, at setting ‘3’, for 30 seconds. The mixture is poured into 150 ml PVA 2% solution, saturated with DCM, and homogenized (Silverson, 1000 rpm for 15 seconds). The resulting emulsion is immediately poured into 1 L DDW and stirred for 30 minutes. Particles are isolated by pouring the mixture over a stack of filters starting at 20 μm size and higher.

### Example 3

### siRNA Formulation Step Activity Scanning—Influence of Emulsification

a) siRNA/DOTAP Emulsions

### Methods

### 1. Transfection

siRNA complexes (2 μl 20 μM siRNA per group) were formed using 5.6 μl DOTAP/Cholesterol (9:1) 1 mg/ml in 120 μl water. 1 ml of various solvents were added to the water phase and mixed at 10 krpm for 60 seconds. The solvents used were:
Dichloromethane
Chloroform
EthylAcetate
Cyclohexane

Subsequently the emulsions were spun for 4 minutes at 10 krpm, and the organic phase was removed and evaporated under vacuum. The water phase was left in the hood at room temperature to ‘airate’ out residual solvent. In a follow-up experiment all solvents (including the water phase) were evaporated in vacuum. Specific anti-luciferase siRNA was compared in parallel with scrambled (non-coding) siRNA.

400 μl DMEM (FBS free, with 5 μg/ml doxycyclin) was added to the resulting residues and vortexed thoroughly (siRNA at 100 nM concentration). HR5CLII doxyclin-induced luciferase expressing cells were plated at 10⁴ cells/well in clear bottom black 96-well plates and incubated 24 hours prior to transfection. 100 μl of the siRNA at 100 nM concentration was added to 3 wells with HR5CL11 cells. 300 μl DMEM was then added to dilue the siRNA concentration to 25 nM. 100 μl of siRNA at 25 nM concentration was added to another 4 wells with HR5CLII cells.

### 2. Toxicity and Luciferase Assay

24 hours after transfection all media was removed. The cells were first incubated with Cell Titre Blue™ to conduct toxicity assessments. After incubation for 1.5 hours, the plate was read using fluorescence (λₑₓ=560 nm, λₑₘ=590 nm). The cells were then washed once with PBS and lysed using Bright-Glo™ lysis buffer. Luciferin was added and the level of luciferase expression was measured using chemiluminescence.

### Results

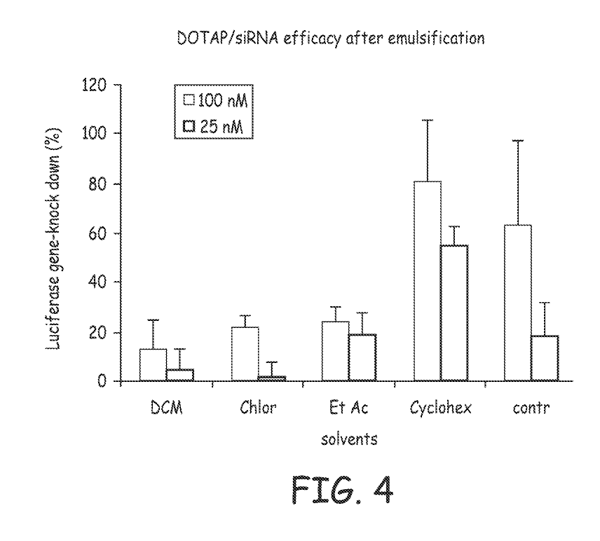

siRNA induced silencing effects were seen both from products in the water phase as well as from the residue extracted into the organic phase. This seemed to indicate that not only does DOTAP partition into the organic phase, it is also able to transport siRNA with it. Except for toluene/cyclohexane, where this effect was not seen, overall efficiency was lower than 50% gene knock-down. FIG. 4.

The effects of emulsification into an organic solvent where also investigated for each phase by itself and compared to the combined system. Upon emulsification the samples were spun in order to separate the two phases. The phases were dried, taken up in cell medium separately and tested for any gene-knockdown activity. The results are presented in Table 1 below:

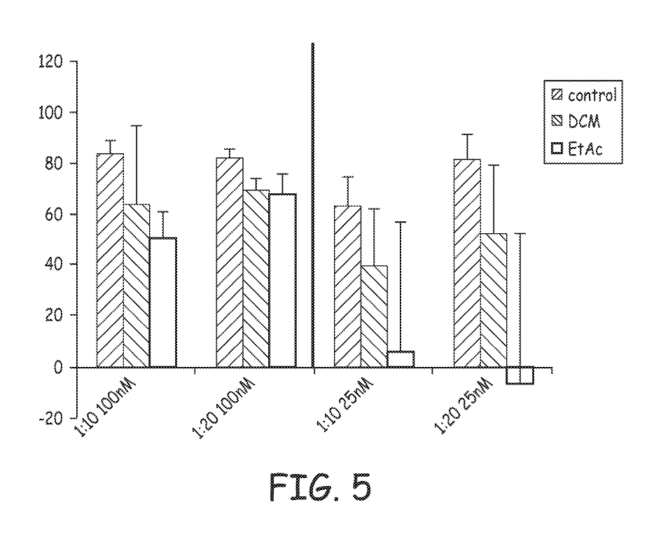

TABLE 1

| 100 nM siRNA | Water Phase | Organic Phase | Combined |
|---|---|---|---|
| DCM | 26 | 16 | 63 |
| Chloroform | 39 | −8 | 22 |
| Ethyl Acetate | 29 | 34 | 51 |
| Toluene | 84 | 23 | 81 (cyclohexane) |
| Control | 88 | | 83 |

Whereas the combined phases showed some transfection, both with ethyl acetate and dichloromethane, the total transfection was at or below 50%. Emulsion into toluene or cyclohexane did not result in lower activities for the combined system. Also very little activity was seen in those organic phases. When the DOTAP/Cholesterol to siRNA ratio was increased from 1:10 (w/w) to 1:20, the following results in FIG. 5 were obtained (after emulsification the phases were not separated, all solvents were removed in vacuum and residues were reconstituted in cell medium by thorough vortexing). These data indicate that emulsifying siRNA/DOTAP solutions leads to a reduction in efficacy due to the fact that DOTAP partitions into the organic phase.

b) siRNA/N-ter Emulsions

### Methods

### 1 Transfection

siRNA complexes (1.5 μl 20 uM siRNA per group) were formed with 3.75 μl N-ter in 50 μl N-ter specific buffer. siRNA complexes were typically prepared at larger amounts and then divided in 50 μl portions. 0.4 ml of various solvents were added to the water phase and mixed at 10 krpm for 60 seconds. The solvents used were:
Dichloromethane
Chloroform
EthylAcetate
Cyclohexane

Subsequently the emulsions were spun 4 minutes at 10 krpm, and the organic phase was removed and evaporated under vacuum. The water phase was left in the hood at room temperature to ‘airate’ out residual solvent. In a follow-up experiment all solvents (including the water phase) were evaporated in vacuum. Specific anti-luciferase siRNA was compared in parallel with scrambled (non-coding) siRNA.

300 μl DMEM (with FBS and 5 μg/ml doxycyclin) was added to the resulting residues and vortexed thoroughly (resulting in siRNA at 100 nM concentration). HR5CLII doxyclin-induced luciferase expressing cells were plated at 104 cells/well in clear bottom black 96-well plates and incubated 24 hours prior to transfection. 100 μl of the 100 nM siRNA solution was added to 2 wells with HR5CLII cells. DMEM was then added to the 100 nM siRNA solution to form a solution containing siRNA at 25 nM of which 100 μl was added to 3 wells. 200 μl DMEM was added to the 25 nM solution to generate a solution containing siRNA at approx. 8 nM of which 100 μl was added to 3 more wells with HR5CL11 cells.

### Toxicity and Luciferase Assay

24 hours after transfection all media was removed. The cells were first incubated with Cell Titre Blue™ reagent to conduct toxicity assessments fluorescence using a SpectraMax plate reader. After incubation for 1.5 hours the plate was read using (ëₑₓ=560 nm, ëₑₘ=590 nm). The cells were then washed once with phosphate buffered saline (PBS) and lysed using Bright-Glo™ lysis buffer. Luciferin was added and the level of luciferase expression was measured using chemiluminescence.

### Results

Table 2 shows similar results as described earlier for DOTAP emulsions. In these studies more activity was seen in the organic phase. One hypothesis is that Nter partitions even more into the organic phase, transferring some of the siRNA. In the water phase complexes are ‘stripped down’ from Nter to the point they are no longer able to transfect cells. Only 100 nM results are presented as lower concentrations gave less transfection and effects were less clear to discern.

TABLE 2

| 100 nM siRNA | Water Phase | Organic Phase | Combined |
|---|---|---|---|
| DCM | −35 | 32 | 44 |
| Chloroform | −4 | 42 | 45 |
| Ethyl Acetate | −31 | 40 | 47 |
| Toluene | 27 | 24 | 79 (cyclohexane) |
| Control | 91 | | 79 |

### c) Addition of Proteins to Protect Nter/siRNA Complexes in Emulsions

### Methods

SiRNA complexes (1.5 μl 20 μM siRNA per group) were formed with 3.75 μl N-ter in 50 μl N-ter specific buffer. Typically 6 to 9 times of the amounts were mixed and the resulting siRNA/Nter complexes in buffer were then divided in 50 μl portions. IgG (lyophilized, Lampire, reconstituted in slightly acidic PBS with drop of HCl), was put on BioRad™ desalting column and eluted with 10 mM phosphase (Ps)/no NaCl. Using a centrifuge filter the protein solutions were concentrated to approximately 20 mg/ml. Alternatively, IgG was used in regular phosphate buffered saline (PBS) at 20 mg/ml.

50 μl of the following solutions was added to the samples:
IgG at 20 mg/ml in phosphate buffered saline (PBS)
IgG at 20 mg/ml in 10 mM Ps (phosphase, no NaCl)

0.4 ml of various solvents were added to the water phase and mixed at 10 krpm for 60 seconds. The solvents used were:
Dichloromethane
Chloroform
EthylAcetate
Cyclohexane

Subsequently the emulsions were spun 4 minutes at 10 krpm, the organic phase was removed and evaporated under vacuum. All solvents (including the water phase) were evaporated in vacuum. Specific anti-luciferase siRNA was compared in parallel with scrambled (non-coding) siRNA.

600 μl DMEM (with FBS and 5 ug/ml doxycyclin) was added to the resulting residues and vortexed thoroughly (siRNA at 50 nM concentration).

HR5CL11 doxyclin-induced luciferase expressing cells were plated at 10⁴ cells/well in clear bottom black 96-well plates and incubated 24 hours prior to transfection. 100 μl of the 50 nM siRNA solution was added to 4 wells with HR5CL11 cells. 200 μl DMEM was then added to the 50 nM siRNA solution to obtain siRNA at 25 nM. 100 μl of the siRNA at 25 nM concentration was added to 4 more wells with HR5CL11 cells.

### Results

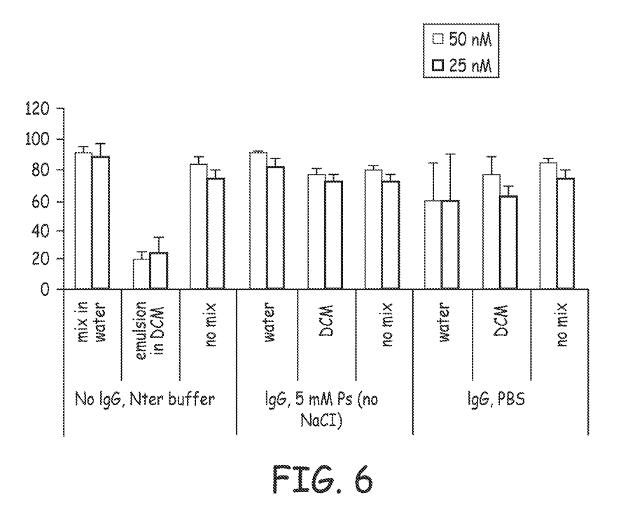

The results in FIG. 6 demonstrate that, in the absence of protein, a significant amount of transfection efficiency is lost. However, when IgG solution is added to the siRNA/N-ter complex a stabilization took place.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as “arranged”, “arranged and configured”, “constructed and arranged”, “constructed”, “manufactured and arranged”, and the like.

In the specification and claims, the “about” is use to modify, for example, the quantity of an ingredient in a composition, concentration, volume, process temperature, process time, yield, flow rate, pressure, and like values, and ranges thereof, employed in describing the embodiments of the disclosure. The term “about” refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods, and like proximate considerations. The term “about” also encompasses amounts that differ due to aging of a formulation with a particular initial concentration or mixture, and amounts that differ due to mixing or processing a formulation with a particular initial concentration or mixture. Where modified by the term “about” the claims appended hereto include equivalents to these quantities.

All publications and patent applications in this specification are indicative of the level of ordinary skill in the art to which this invention pertains. All publications and patent applications are herein incorporated by reference to the same extent as if each individual publication or patent application was specifically and individually indicated by reference.

This application is intended to cover adaptations or variations of the present subject matter. It is to be understood that the above description is intended to be illustrative, and not restrictive.

We claim:

1. A composition for delivering small inhibitory nucleic acid (siRNA) to a target cell, the composition comprising siRNA and a stabilizing protein.

2. The composition of claim 1, wherein the stabilizing protein is selected from the group consisting of Immunoglobulin G (IgG), a fragment antigen-binding (Fab fragment), transferrin, bovine serum albumin (BSA) or human serum albumin (HSA).

3. The composition of claim 1, wherein the siRNA is complexed with a carrier.

4. The composition of claim 3, wherein the carrier comprises a peptide carrier.

5. The composition of claim 4, wherein the peptide carrier is a polycationic or amphipathic cell penetrating peptide.

6. The composition of claim 5, wherein the peptide carrier is selected from the group consisting of N-ter and PTD-DRBD.

7. A composition for delivering small inhibitory nucleic acid (siRNA) to a target cell, the composition comprising siRNA combined with a protein, wherein the protein is cross-linked to form a proteinaceous controlled release matrix.

8. A method of making a composition for delivering a nucleic acid reagent to a target cell, the method comprising:
combining the nucleic acid reagent, one or more stabilizing proteins, and one or more matrix forming polymers in an emulsion with a dispersed phase and a continuous phase;
extracting the solvent from the sipersed phase into the extraction phase to form microparticles containing the nucleic acid reagent, one or more stabilizing proteins and one or more matrix forming proteins.

9. The method of claim 8, wherein the nucleic acid reagent comprises siRNA.

10. The method of claim 8, wherein the nucleic acid reagent comprises a nucleic acid complexed with a carrier.

11. The method of claim 10, wherein the carrier comprises a peptide carrier.

12. The method of claim 8, wherein the stabilizing protein is selected from the group consisting of Immunoglobulin G (IgG) and a fragment antigen-binding (Fab fragment), transferrin, bovine serum albumin (BSA) and human serum albumin (HSA).

13. A method of delivering a small inhibitory nucleic acid (siRNA) to a target cell, the method comprising administering the composition of claim 1 to a target cell.

14. A method of making a composition for delivering a nucleic acid reagent to a target cell, the method comprising:
combining nucleic acid reagent with a protein in an aqueous solution;
adding a phase separation agent to the aqueous solution, wherein the phase separation agent causes the siRNA and protein to precipitate out of solution to form particles; and
cross-linking the protein to form a proteinaceous controlled release matrix.

15. The method of claim 14, wherein the nucleic acid reagent comprises siRNA.

16. The method of claim 14, wherein the nucleic acid reagent comprises a nucleic acid complexed with a carrier.

17. The method of claim 16, wherein the carrier comprises a peptide carrier.

18. The method of claim 17, wherein the peptide carrier comprises N-ter.
